(19) United States
(12) Patent Application Publication
(10) Pub. No.: US 2003/0215812 A1
(43) Pub. Date: Nov. 20, 2003

(54) Gene encoding beta-mannanase, enzyme preparation and uses thereof

(76) Inventors: Yan-He Ma, Beijing (CN); Yan-Fen Xue, Zhixinbeili (CN)

Correspondence Address: SHANKS & HERBERT, 1033 N. FAIRFAX STREET, SUITE 306, ALEXANDRIA, VA, 22314, US

(21) Appl. No.: 10/147,324

(22) Filed: May 17, 2002

(51) Int. Cl. C12Q 1/68; C07H 21/04; C12N 9/24; C12N 1/21; C12P 21/02; C12N 15/74

(52) U.S. Cl. 435/6; 435/69.1; 435/200; 435/252.31; 435/320.1; 536/23.2

(57) ABSTRACT

The present invention relates to genes coding for novel β-mannanases produced by the alkaliphilic Bacillus sp. N16-5, recombinant vectors and host cells containing such gene. The present invention also provides an enzyme preparations containing such enzymes produced by alkaliphilic Bacillus sp. N16-5 as well as the use of such preparations. The β-mannanases of the present invention can effectively hydrolyze many plant polysaccharides, such as konjak polysaccharide to produce a series of oligosaccharides.

### FIELD OF THE INVENTION

The present invention relates to genes encoding novel β-mannanase, the preparation of a recombinant enzyme coded by the gene and uses thereof. Particularly, the present invention relates to nucleic acid molecules encoding β-mannanase produced by alkaliphilic Bacillus sp. N16-5, a recombinant vector and host cells containing said gene as well as methods for producing and using the polypeptides.

### BACKGROUND OF THE INVENTION

β-mannanase (EC 3.2.1.78) is an enzyme that cleaves the β-1,4-mannosidic linkages in mannan, glucomannan, galacto-mannan, and galacto-glucomannan (Tipson et al., 1976). Mannan is one of the major hemicellulose constituents of plants. Some kinds of plants such as endosperus of copra and ivory palm nuts, beans of guar, locust and coffee, and roots of konjak contain a lot of mannan. Potential uses of β-mannanase have been shown to promote pulp bleaching ability in the manufacture of kraft pulp (Buchert et al. 1997; Christgau et al., 1998; Khanongnuch et al. 1999, WO), to produce oligosaccharides from hemicellulose as one of the best growth factors for Bifidobacterium sp. (Kobayashi et al. 1983), to hydrolysis galactomannans in the massive hydraulic fracturing of oil well (McCutchen et al. 1996) and to provide excellent cleaning performance on food and cosmetic stains in detergent industry (Philippe et al. 2000, Sreekrishna et al., 2000).

β-mannanase has been isolated from some microorganisms, such as Bacillus, Aeromonas, Pseudomonas, Caldocellum, Streptomyces, Piromyces, Aspergillus and Trichodema. Some patents and literatures regarding the gene of mannanase have been reported in recent years. Most of them were neutral or acidic. Alkaline β-mannanases have not been intensively investigated, although the properties of alkaline β-mannanase provide obvious advantages for enzyme to be used in the applications such as the manufacture of kraft pulp and detergent industry. In addition, hemicellulose swells better in alkaline condition, in that hydrolysis of hemicellulose using alkaline enzyme is more effective. Some Bacillus have been reported to produce alkaline β-mannanases. JP-0304706 described a mannanases with optimal activity at pH8-10 from Bacillus sp. JP-63036774 described a mannanase produced by Bacillus sp. FERM P-8856 at alkaline pH. WO 91/18974 describes a mannanase active at an extreme pH and temperature. Bacillus sp. AM001 produced three extrocellular mannanases with optimal activity at pH8-9 and 60-65° C. (Akino et al. 1987; 1988, 1996).

Alkaliphilic Bacillus strain often produces various alkaline enzymes, such as alkaline CMCase, protease, and amylase (Horikoshi, 1996). We isolated a strictly alkalophilic Bacillus sp. N16-5, which had an optimum pH 10 for growth and produced significant amounts of extracellular β-mannanases (Ma et al. 1991), which showed an optimal activity at pH 9-10 and 70° C. and effectively hydrolyzed many plant polysaccharides such as konjak polysaccharide. The pH and temperature optimum and stability of Bacillus sp. N16-5 mannanase is unique to other β-mannanase characterized so far.

### OBJECTS OF THE INVENTION

One object of the present invention is to provide nucleic acid molecules coding for novel β-mannanase.

The second object of the present invention is to provide a recombinant vector containing said β-mannanase gene and a recombinant strain containing said β-mannanase gene.

The third object of the present invention is to provide a process for producing the β-mannanase, and using the same to hydrolyze mannan effectively.

### SUMMARY OF THE INVENTION

The present invention provides a DNA sequence coding for new β-mannanase produced by an alkaliphilic Bacillus sp. N16-5, comprising the amino acid sequence of SEQ ID NO: 2.

According to the DNA sequence of the present invention, wherein, the said DNA sequence comprises the nucleotide sequence of SEQ ID NO: 1.

The present invention also provides a recombinant vector and a recombinant host containing the above-mentioned DNA sequence.

According to another aspect of the invention, it provides a process for enzyme preparations from the growth of such hosts that contain the expressed mannanase, and the use of such enzyme preparations including production of manno-oilgosaccharides.

### BRIEF DESCRIPTION OF THE FIGURES

FIG. 1 shows the structure of the recombinant plasmid pMAN1 containing β-mannanase gene of the present invention.

FIG. 2 is the DNA sequence of β-mannanase of the present invention.

FIG. 3 is the deduced amino acid sequence of β-mannanase of the present invention.

### DETAILED DESCRIPTION OF THE INVENTION

The present invention is accomplished by the following findings made by the present inventors, that is, the alkaliphilic Bacillus sp. N16-5 can produce a large amount of alkaline β-mannanases, and the enzyme is able to hydrolyze plant polysaccharides, such as konjak powder.

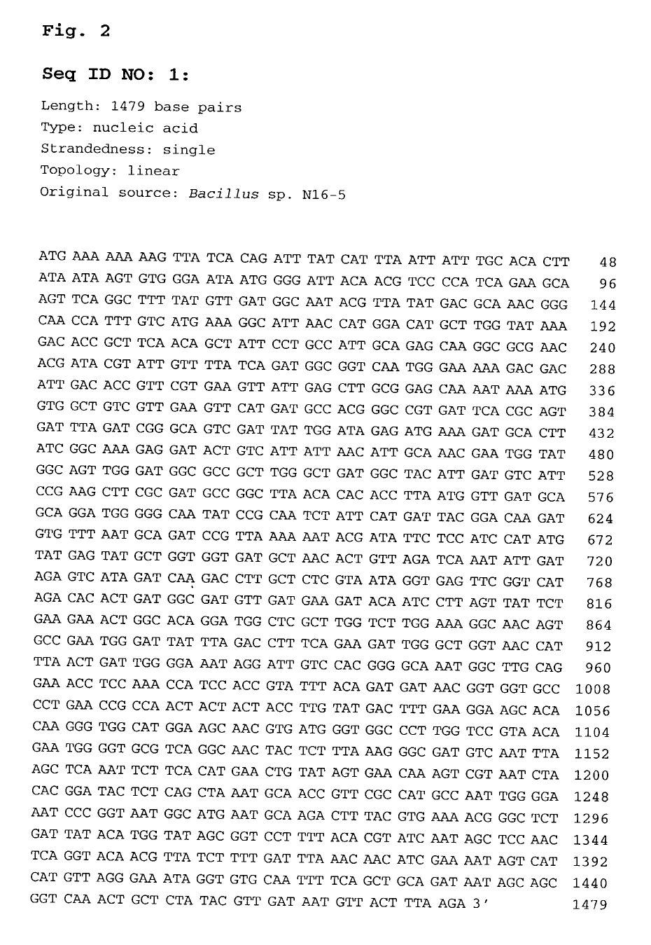

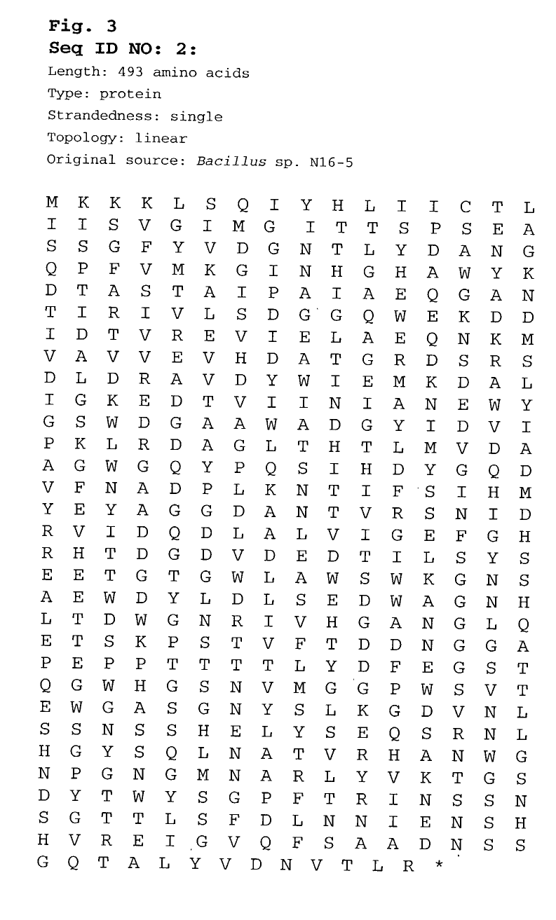

The present invention provides a β-mannanase gene obtained from an alkaliphilic Bacillus sp. N16-5. The gene contains an ORF of 1479 bp and its nucleotide sequence is shown in FIG. 2 (SEQ ID NO: 1). The gene codes a polypeptide consisting of 493 amino acids. The amino acid sequence of the polypeptide is shown in FIG. 3 (SEQ ID NO: 2). By using the conventional methods, recombinant vector and host comprising the said gene are obtained. The above-mentioned recombinant host such as E. coli DH5αMAN2 was cultivated under the suitable conditions to express the said β-mannanase gene. A clear zone formed around the colony on the agar plate with knojak proves that the recombinant strain can express the β-mannanase activity. Therefore, the present invention provides a possibility to produce the β-mannanase of the present invention by other host or at other condition.

The properties of the β-mannanase of the present invention are different from those of the known β-mannanases. The said β-mannanase has an optimal pH of 9-10 for activity and stable pH range of 8.5-10, the optimum temperature of 70° C. for activity, and pI of about 4.3. The pH optimum and stability of the said mannanase exceed the values found for other mannanase characterized so far. The amino acid sequence similarity of the said β-mannanase with the previously reported β-mannanases is less than 60%. Thus, the β-mannanase of the present invention is a novel β-mannanase.

The expression product of the said gene hydrolyzes plant polysaccharide to produce manno-oligosaccharides effectively. The concentration of the substrate is in the range of 5-15%, the reaction is performed at temperature of 40-60° C., pH of 9-10 for 8-24 hr. After the reaction, the pH of the reaction mixture was adjusted to 5-6 by adding acetic acid, citric acid or hydrochloric acid. Manno-oligosaccharides slurry is obtained after decolorization and filtration. The manno-oligosaccharides conversion rate is larger than 80%, and the total yield is more than 70%.

The term “β-mannanase” is defined as an enzyme that cleaves the β-1,4-mannosidic linkages in mannan, glucomannan, galacto-mannan, and galacto-glucomannan. β-mannanase activity was assayed with locust bean gum as the substrate by measuring the reducing sugars liberated during the hydrolysis of mannan as described previously (Akino et al., 1987). One unit of activity was defined as the amount of enzyme catalyzing the production of 1 μmol of the reducing sugar per min using mannose as the standard.

An amino acid sequence that is an “equivalent” of a specific amino acid sequence is meant to be an amino acid sequence that is not identical to the specific amino acid sequence, but rather contains at least some amino acid changes (deletion, substitutions, inversions, insertions, etc.) that do not essentially affect the biological activity of the protein as compared to a similar activity of the specific amino acid sequence, when used for a desired purpose. Preferably, an “equivalent” amino acid sequence contains at least 80%-99% identity at the amino acid level to the specific amino acid sequence (i.e., that of SEQ ID NO: 2).

A nucleic acid molecule that encode a polypeptide of the present invention is derived from a variety of sources. These sources include genomic DNA, cDNA, synthetic DNA and combinations thereof. Preferably, the nucleic acid sequences have a degree of homology to the polypeptide coding sequence of SEQ ID NO: 1 of at least more than 80% homology. A nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated.

The recombinant vector may be any vector (e.g., a plasmid or virus) comprising a nucleic acid sequence of the present invention. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. On the other hand, the vector may be integrated into the genome of the host cell by non-homologous recombination. The choice of the vector will typically depend on the compatibility of the vector with the host cell into which the vector is to be introduced. The vectors may be linear or closed circular plasmids. The procedures used to ligate the elements described above to construct the recombinant expression vectors of the present invention are well known to one skilled in the art (Sambrook et al., 1989).

The term “host cell” means a host in which the activity of the said enzymes is depressed, deficient or absent when compared to the wild type. The host cell may be a unicellular microorganism, e.g., a prokaryote, or a non-unicellular microorganism, e.g., a fungal cell and a yeast cell. The recombinant host cells comprise a nucleic acid sequence of the invention, which is advantageously used in the recombinant production of the polypeptides.

Accordingly, the mannanase encoding sequences may be operably linked to any desired vector and introduced into an appropriate host cell by any of a variety of suitable means. After the introduction of the vector, recipient cells are grown in a selective medium, which selects for the growth of transformed cells. Expression of the cloned gene sequence(s) results in the production of the desired protein, or in the production of a fragment of this protein.

The term “hybridization” means hybridization under conventional hybridization conditions, preferably under stringent conditions such as described by, e.g. Sambrook et al. (1989). As hybridization probe, e.g. nucleic acid molecules can be used that have exactly or substantially the same nucleotide sequence indicated in the FIG. 2 or fragments, derivatives and allelic variants of said sequence. Fragments are understood to be parts of nucleic acid molecules long enough to code for the described protein or a biologically active fragment thereof. The fragments used as hybridization probes can also be synthetic fragments obtained by conventional synthesis techniques and the sequence of which is substantially identical to that of the nucleic acid molecules of the invention. The term “derivative” means that the nucleotide sequences of these molecules differ from the sequences of the above-described nucleic acid molecules in one or more positions and are highly homologous to said sequence. Homology is understood to refer to a sequence identity of at least 70%, preferably more than 80%. Furthermore, homology means that the respective nucleotide sequences or encoded proteins are functionally and/or structurally equivalent. The deviations from the nucleic acid molecules described above can be the result of deletion, substitution, insertion, addition or combination.

The mannanase encoding sequences described herein may be fused in frame to other sequences so as to construct DNA encoding a fusion protein. The result is a protein that contains a mannanase of the invention. The carrier polypeptide needs not have enzymatic activity or its activity may be inactivated.

An enzyme preparation is meant to be a composition containing medium previously used to culture a desired microbe(s) and said mannanase that have been released from the microbial cells into such medium during the culture, or downstream processing steps.

The invention provides methods for producing enzyme preparations. Spent medium from the growth of the recombinant hosts, or purified enzymes therefrom, can be used as the source of the enzyme preparations of the invention in the desired application. The enzyme preparations of the invention may be provided as a liquid or as a solid, for example, in a dried powder or granular or liquid form, especially nondusting granules, or a stabilized liquid, or the enzyme preparation may be otherwise concentrated or stabilized for storage or use. The enzyme preparations of the invention can be adjusted to satisfy the requirements of specific needs in various industrial applications.

Generally, the present enzyme preparations are useful for degradation of mannan-containing substrates. Owing to their activity at alkaline pH values, the β-mannanase of the invention are well suited for use in a variety of industrial processes, in particular the enzyme finds potential applications in detergents and in the manufacture of kraft pulp, but it may also be useful in the preparation of manno-oligosaccharide. The present invention comprises a method for enzymatically treating plant biomass under conditions of temperature (50-60° C.) and pH 8-10 for a desired time, such as, for example, 8 hours.

The present invention will be further described by the following examples. It should be understood that these examples are used to explain, not to limit the scope of the present invention.

### EXAMPLE 1

Extraction of Genomic DNA from Alkaliphilic Bacillus sp. N16-5

The alkaliphilic Bacillus sp. N16-5 was isolated from the Lake Wuduzuo, Inner Mongolia, China. 20 grams of the fresh Bacillus sp. N16-5 cells were suspended in 10 ml 50 mM Tris buffer (pH 8.0). A small amount of lysozyme and 8 ml 0.25 mM EDTA (pH 8.0) were added thereto. After mixed homogeneously, the mixture was incubated for 20 minutes at 37° C. Then, 2 ml of 10% SDS was added, and the mixture was incubated for 5 minutes at 55° C. The mixture was extracted with the same volume of phenol and a mixture of chloroform: isoamyl alcohol (24:1 v/v), respectively. The supernatant was collected, and two volume of ethanol was added thereto. The precipitate of DNA was collected, and was washed by 70% ethanol and absolute ethanol respectively. The precipitate was solved in 0.5 ml TE buffer solution (pH 8.0, 10 mM Tris, 1 mM EDTA), and treated with 3 μl 10 mg/ml RNase to remove, RNA. The genomic DNA was purified as described by Sambrook et al. 1989, and solved in de-ionized water. The measured result of the DNA solution by ultraviolet spectrometer was A260/A280=1.98, A260/A230=2.18.

### EXAMPLE 2

The Clone of β-Mannanase Gene

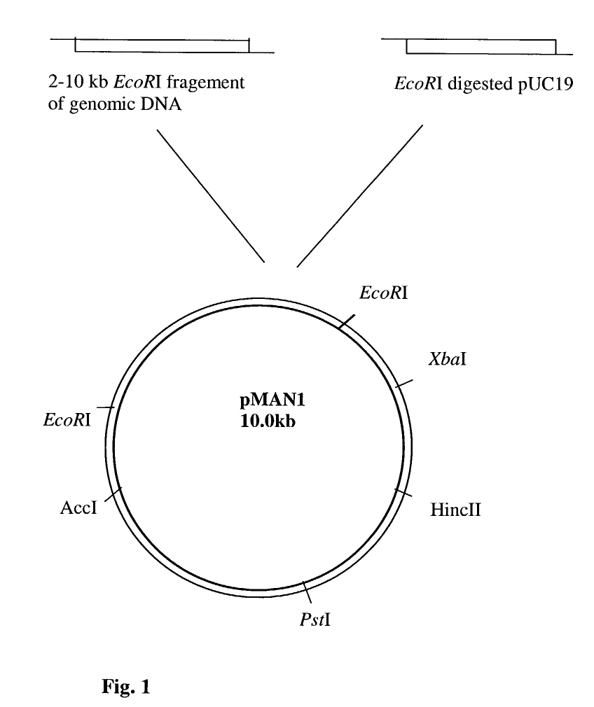

10 μl (about 50 μg DNA) of the total DNA obtained in Example 1 was partially digested with EcoRI (Boehringer Mannheim, Germany). After agarose electrophoresis, 2-10 kb DNA fragements were obtained. 2 μl (5μg) EcoRI digested DNA fragement and 1 μl (1 μg) EcoRI digested pUC19 vector were ligated in a 20 μl reaction system which comprising 2 μl (10× buffer), 1 μl T4 DNA ligase. The mixture was reacted at 16° C. for 16 hours and transformed into the competent E. coli DH5α. Transformed cells were grown on LB agar plate supplemented with ampicillin, X-gal and IPTG at 37° C. for 16 h. All white colonies were transferred to a LB plate containing 1% konjak mannan and ampicillin. The clones expressing β-mannanase activity were identified by the formation of clear halos around colonies using the Congo red staining (Teathor and Wood, 1982). The recombinant vectors were extracted from the positive bacteria colony, and were digested with single and multiple restriction enzyme. The electrophoresis results showed that the 8 kb DNA fragements were inserted into the pUC19 vector. The recombinant vector containing the DNA insert was called pMAN1 (FIG. 1), and the recombinant E. coli comprising the pMAN1 was called E. coli DH5αMAN1.

The recombinant plasmid pMAN1 could effectively transfer E. coli to express the β-mannanase activity. Southern hybridization was done to identify that cloned gene originated from Bacillus sp. N16-5. The 8 kb probe was prepared from the EcoRI fragment in pMAN1, labeled with DIG-dUTP using DIG DNA labeling kit (Boehringer Mannheim, Germany) and hybridization, washing, and development procedure were followed according to the manufacturer's manual. The results proved that the inserted DNA in the recombinant vector pMAN1 was from the chromosomal DNA of the alkaliphilic Bacillus sp. N16-5.

### EXAMPLE 3

The Sub-Cloning and Sequencing of β-Mannanase Gene

The plasmid (pMAN1) was digested with restrict enzymes, such as AccI, HindIII, PstI, EcoRI and XbalI. According to the methods as mentioned in Example 2, the obtained digested DNA fragements were ligated to pUC19 to form a series of sub-clone vectors. These sub-clone vectors were transferred into the E. coli to obtain a series of recombinant E. coli. Their enzyme activities were measured. The results showed that the recombinant E. coli comprising the AccI-HincII fragment showed mannanase activity. The size of the AccI-HincII fragment was about 2.0 kb. The recombinant vector comprising the AccI-HincII fragment was called pMAN2, and the recombinant E. coli containing the pMAN2 was called E. coli DH5αMAN2. The nucleotide sequence of both strands of the DNA insert were determined by the Sanger dideoxy chain termination methods using an ABI 377S DNA sequencer (Applied Biosystems).

A total of 2058 bp nucleotides were sequenced (FIG. 2). And one open reading frame (ORF) corresponding to an initiation ATG codon beginning at position 403 of the determined sequence and ending with a TAG codon at 1882 was found. The ORF (manA) corresponding to mannanase (ManA) consists of 1479 bp and codes for a protein of 493 amino acid residues with an estimated molecular mass of 54215 Da.

### EXAMPLE 4

The Purification and Characteristics of the β-Mannanase

The cell of the recombinant bacteria E. coli DH5αMAN2 was suspended in a 10 mM glycine buffer solution (pH 9.6). The cells were broken by frozen-thaw method. The supernatant fluid obtained by centrifugation was the crude enzyme solution of recombinant β-mannanase. The β-mannanase was purified by DEAE-Sephadex ion exchange chromatography, hydroxyapatite hydrophobic chromatography and preparation electrophoresis. The obtained enzyme preparation showed a sigle band on SDS-PAGE. The characteristics of this recombinant β-mannanase were measured by standard methods. The molecule weight of the enzyme measured by SDS-PAGE was 51,000 Dolton, which was very similar to the theoretical value (54,000 Dolton). The isoelectric point (pI) measured by PAGEIEF was 4.3. The optimum pH for enzyme activity was 9.0-10, and the optimum temperature for enzyme activity was 70° C. The HPLC analysis showed that the recombinant β-mannanase can hydrolyze knojak polysaccharide to produce a series of oligasaccharides.

### EXAMPLE 5

The Production of Oligosaccharides by β-Mannanase

1.8 liters of water, 4.8 grams of Na₂CO₃, 3 grams of NaHCO₃, 1.8×10⁴ units of β-mannanase and 180 grams of konjak powder were added into the 2.5 liter reactor. The mixture was incubated at 55° C. for 16 hours. The reaction was ended by adjusting pH value of the mixture to 5.5-6.0 with hydrochloric acid. 10 grams of activated carbon particles were added thereto, and the temperature was raised to 100° C. and kept at this temperature for 5 minutes. Then, the temperature was cooled to 70° C., and the reaction mixture was filtered. The results showed that the C₂₋₈ oligosaccharides amounted more than 80% of the total sugar, the oligosaccharide conversion rate was more than 80%, and the recovery of oligosaccharide was more than 70%. The composition of the oligosaccharide obtained in this example was shown in the following Table.

TABLE
The composition of the manno-oligosaccharides

| Reaction | Total sugar | Oligosaccharides | Percentage | Conversion |
|---|---|---|---|---|
| Time (h.) | (g/100 ml) | (g/100 ml) | of C₂₋₈ | Rate |
| 0 | 9.4 | / | / | 0 |
| 4 | 9.2 | 1.725 | 46% | 19% |
| 8 | 9.7 | 6.894 | 70% | 71% |
| 16 | 9.6 | 8.178 | 81% | 85% |

References

Akino et al.: Agric Biol Chem 52, 773-779, 1987

Akino, T. Et al.: JP-63056289, 1988.

Akino, T. Et al.: JP-08051975, 1996.

Buchert et al.: U.S. Pat. No. 5,661,021, Aug. 26, 1997;

Christgau et al.: U.S. Pat. No 5,795,764, Aug. 18, 1998

Horikoshi: FEMS Microbiol Rev 18, 259-270, 1996,

Kobayashi et al.: Proceedings of the 4^{th} RIKEN Symposium on Intestinal Flora, pp69-90, Japan Scientific Societies Press, Tokyo, 1984;

Khanongnuch rt al.: Biotechnol Lett. 21(1): 61-63, 1999

Sreekrishna et al.: U.S. Pat. No. 6,060,299, May 9, 2000

McCutchen et al.: Biotechnol Bioeng 52:332-339, 1996

Mendoza et al., World J. Microbiol Biotech., vol. 10, No. 5, pp. 551-555, (1994).

Philippe et al.: EP1059351, 2000

Ratto, M. Et al.: Biotechnol. Letters, 10:661-664;

Sambrook et al.: Molecular Cloning: a Laboratory Manual, 2^{nd} edn. Cold Spring Harbor, N.Y., 1989

Talbot et al., Appl. Environ. Microbiol vol. 56, No. 11, pp. 3505-3510 (1990).

Teathor and Wood: Appl Environm Microbiol 43:777-780, 1982

Tipson et al.: Advances in Carbohydrate Chemistry and Biochemistry, 32:299-316, Academic Press, New York, 1976

What we claimed is:

1. A nucleic acid molecule encoding β-mannanase produced by an alkaliphilic Bacillus sp. N16-5, selected from the group consisting of:
(a) nucleic acid molecules encoding a polypeptide comprising the amino acid sequence as depicted in FIG. 3 (SEQ ID NO: 2);
(b) nucleic acid molecules comprising the coding sequence of the nucleotide sequence as depicted in FIG. 2 (SEQ ID NO: 1).
(c) nucleic acid molecules the coding sequence of which differs from the coding sequence of a nucleic acid molecule of (a) and (b) due to the degeneracy of the genetic code; and
(d) nucleic acid molecules hybridizing to a molecule of (b); and encoding a polypeptide having β-mannanase activity and having an amino acid sequence which shows at least 80% identity to a sequence as depicted in FIG. 3 (SEQ ID NO: 2).

2. A recombinant vector containing a nucleic acid molecule of claim 1.

3. The vector of claim 2, in which the nucleic acid molecule is operably linked to expression control sequences allowing expression in prokaryotic or eukaryotic host cells.

4. The recombinant vector of claim 2, wherein the recombinant vector comprises a recombinant plasmid pMAN1 or a recombinant plasmid pMAN2 comprising the nucleic acid molecule of claim 1.

5. A host cell transformed with the nucleic acid molecule of claim 1 or with a vector of any one of claims 2 to 4.

6. The host cell of claim 5, wherein the strain comprises the E. coli DH5αMAN1 or the E. coli DH5αMAN2.

7. A process for the production of a polypeptide having mannanase activity comprising the steps of culturing the host cell of claims 5 or 6 and recovering the polypeptide from the cells or the culture medium.

8. An enzyme preparation comprising the polypeptide having mannanase activity comprising the amino acid sequence of claim 1.

9. An oligonucleotide specifically hybridizing under stringent conditions to any nucleic acid molecule of claim 1.
